(19) United States
(12) Patent Application Publication
(10) Pub. No.: US 2024/0005589 A1
(43) Pub. Date: Jan. 4, 2024

(54) ULTRASONIC IMAGE PROCESSING DEVICE AND ULTRASONIC IMAGE PROCESSING PROGRAM

(71) Applicant: FUJIFILM Healthcare Corporation, Kashiwa-shi (JP)

(72) Inventor: Toshinori Maeda, Chiba (JP)

(21) Appl. No.: 18/211,483

(22) Filed: Jun. 19, 2023

(30) Foreign Application Priority Data
Jun. 29, 2022 (JP) 2022-104353

(51) Int. Cl. G06T 15/08; G06T 7/00; G06V 10/60

(52) U.S. Cl. CPC G06T 15/08; G06T 7/0012; G06V 10/60; G06T 2207/10136; G06T 2207/30044

(57) ABSTRACT

A change amount information calculation unit calculates change amount information indicating an amount of change Vₙ between signal intensity of a target voxel and signal intensity of a neighboring voxel near the target voxel for each voxel forming ultrasound volume data. An opacity calculation unit calculates opacity α(Xₙ)*g(Vₙ) of each voxel such that opacity of the voxel is reduced with reduction in the amount of change Vₙ in the signal intensity from the neighboring voxel. A rendering processing unit performs volume rendering on the basis of signal intensity Xₙ of each voxel and the calculated opacity α(Xₙ)*g(Vₙ) of each voxel and forms a three-dimensional image.

### CROSS REFERENCE TO RELATED APPLICATION

This application claims priority to Japanese Patent Application No. 2022-104353 filed on Jun. 29, 2022, which is incorporated herein by reference in its entirety including the specification, claims, drawings, and abstract.

### TECHNICAL FIELD

The present specification discloses an ultrasonic image processing device and an ultrasonic image processing program, and in particular, discloses improvement in volume rendering processing on ultrasound volume data.

### BACKGROUND

An ultrasonic image processing device has a function of processing data obtained by transmitting and receiving ultrasonic waves and includes an ultrasound diagnostic device, an information processing device, or the like. Data to be processed by the ultrasonic image processing device are acquired by the ultrasound diagnostic device.

Conventionally, the ultrasound diagnostic device generates a three-dimensional image on the basis of reception signals obtained by transmitting and receiving ultrasonic waves to and from a subject. For example, a three-dimensional image showing a state of a fetus is generated in obstetrics. Here, the three-dimensional image is an image that three-dimensionally or stereoscopically expresses the subject (including a living body) and is physically a two-dimensional image. The three-dimensional image can express, for example, the subject (e.g. fetus) with a sense of depth.

The three-dimensional image is formed by processing ultrasound volume data formed based on the reception signals of the ultrasonic waves. The ultrasound volume data are data in which voxels having data indicating signal intensity of reflected waves from the subject are arranged three-dimensionally (e.g. an azimuth direction, a slice direction, and a depth direction). Conventionally, a technique called volume rendering has been proposed as one of methods of forming a three-dimensional image on the basis of ultrasound volume data (e.g. “Volume Rendering”, Robert A. Drebin, et al., Computer Graphics, Volume 22, Number 4, August 1988, “Multi-Dimensional Transfer Functions for Interactive Volume Rendering”, Joe Kniss, et al., Scientific Computing and Imaging Institute, School of Computing, University of Utah, and, “Variational Classification for Visualization of 3D Ultrasound Data”, Raanan Fattal, Dani Lischinski, School of Computer Science and Engineering, The Hebrew University of Jerusalem).

FIG. 6 is an explanatory diagram of volume rendering. Volume rendering is mainly performed as the following processing. First, a plurality of lines of sight called rays R passing through ultrasound volume data Vol from a certain viewpoint are set. Then, integration processing is performed for each ray R on the basis of signal intensity of each voxel V (V1, V2, . . . , and VN in the example of FIG. 6) on the ray R and a parameter called opacity (opacity value). Thus, a calculated value is obtained. Specifically, integration processing shown by the following Expression 1 is performed for each ray R.

[Math. 1]

Cₙ=Cₙ₋₁+(1−αₙ₋₁)*α(Xₙ)*Xₙ (Expression 1)

In Expression 1, Cₙ denotes a calculated value (integrated value) when calculation is performed up to a voxel n that is an nth voxel on the ray R (as viewed from the viewpoint). In the expression, αₙ denotes an integrated value of the opacity up to the voxel n, and α(Xₙ) denotes the opacity of the voxel n. The integrated value of the opacity for the entire ray R is set to be 1. Xₙ denotes the signal intensity of the voxel n. As shown by Expression 1, in volume rendering processing, the calculated value Cₙ for the ray R is calculated by adding a calculated value Cₙ₋₁ up to a previous voxel n−1 on the ray R and the product of an amount of transmission (denoted by 1−αₙ₋₁) transmitted to the voxel n−1, the opacity α(Xₙ) of the voxel n, and the signal intensity Xₙ of the voxel n. That is, it can be said that an influence of the signal intensity Xₙ of the voxel n on the calculated value Cₙ is larger as the opacity α(Xₙ) of the voxel n is larger.

The integrated value αₙ of the opacity up to the voxel n is calculated by the following Expression 2.

[Math. 2]

αₙ=αₙ₋₁+(1−αₙ₋₁)*α(Xₙ) (Expression 2)

That is, the integrated value αₙ of the opacity up to the voxel n is obtained by adding an integrated value αₙ₋₁ of the opacity to up the voxel n−1 and a value ((1−αₙ₋₁)*α(Xₙ)) calculated on the basis of the opacity α(Xₙ) of the voxel n such that the integrated value an of the opacity for the ray is 1.

One ray R corresponds to one pixel, and a pixel value is calculated based on the calculated value. By calculating pixel values for a plurality of rays R, a three-dimensional image including a plurality of pixels is formed.

JP 5622374 B discloses an ultrasound diagnostic device that generates a three-dimensional image on the basis of ultrasound volume data and allows a user to adjust opacity of (each voxel included in) the ultrasound volume data by using a user interface.

The ultrasound volume data obtained by transmitting and receiving ultrasonic waves to and from the subject may include noise. Examples of the noise include electrical noise randomly generated from an electric circuit or the like of the ultrasound diagnostic device and noise caused by tissue in the subject (e.g. noise caused by a minute object such as a floating object in the amniotic fluid in a case where a target is a fetus). Such noise has a certain level of signal intensity in the voxels forming the ultrasound volume data. Therefore, when volume rendering is performed on the ultrasound volume data including the noise to form a three-dimensional image, noise may also occur in the three-dimensional image due to an influence of the noise.

An object of the ultrasonic image processing device disclosed in the present specification is to, even in a case where ultrasound volume data include noise, reduce an influence of the noise in a three-dimensional image obtained by performing volume rendering on the ultrasound volume data.

### SUMMARY

An ultrasonic image processing device disclosed in the present specification includes: a change amount information calculation unit that calculates change amount information indicating an amount of change between signal intensity of a target voxel and signal intensity of a neighboring voxel near the target voxel for each voxel forming ultrasound volume data obtained by transmitting and receiving ultrasonic waves to and from a subject; an opacity calculation unit that calculates opacity of the voxel on the basis of the change amount information regarding the voxel such that the opacity of the voxel is reduced with reduction in the amount of change in the signal intensity between the voxel and the neighboring voxel, the amount of change being indicated by the change amount information regarding the voxel; and a rendering processing unit that performs volume rendering on the ultrasound volume data for each line of sight on the basis of the signal intensity of each voxel and the calculated opacity of the voxel, obtains pixel values, and forms an ultrasonic image on the basis of a plurality of pixel values for each line of sight.

In the ultrasound volume data, a voxel corresponding to noise has a feature that an amount of change between the signal intensity of the voxel and the signal intensity of a neighboring voxel near the voxel is reduced. With the above configuration, the opacity of the target voxel is calculated to be reduced with reduction in the amount of change in the signal intensity from the neighboring voxel. Therefore, an influence of the signal intensity (noise components) of the voxel V corresponding to the noise on a three-dimensional image is reduced in the volume rendering.

The opacity calculation unit may calculate a correction parameter based on the amount of change for each voxel and correct the opacity of the voxel on the basis of opacity predetermined for the voxel and the correction parameter.

With the above configuration, the opacity of each voxel can be corrected on the basis of opacity predetermined for each voxel (with reference to opacity predetermined for each voxel) such that the opacity is reduced with reduction in the amount of change in the signal intensity from the neighboring voxel.

The opacity calculation unit may calculate the correction parameter by normalizing the amount of change to a value between a predetermined minimum value and a predetermined maximum value, and at least one of the maximum value and the minimum value may be changeable in response to a user instruction.

With the above configuration, the user can adjust at least one of the maximum value and the minimum value, thereby obtaining a desired three-dimensional image in the volume rendering.

The neighboring voxel may be a voxel not adjacent to the target voxel in the ultrasound volume data.

The ultrasound volume data may be obtained by transmitting and receiving an ultrasonic wave to and from a fetus.

An ultrasonic image processing program disclosed in the present specification causes a computer to function as: a change amount information calculation unit that calculates change amount information indicating an amount of change between signal intensity of a target voxel and signal intensity of a neighboring voxel near the target voxel for each voxel forming ultrasound volume data obtained by transmitting and receiving ultrasonic waves to and from a subject; an opacity calculation unit that calculates opacity of each voxel on the basis of the change amount information regarding the voxel such that the opacity of the voxel is reduced with reduction in the amount of change in the signal intensity between the voxel and the neighboring voxel, the amount of change being indicated by the change amount information regarding the voxel; and a rendering processing unit that performs volume rendering processing on the ultrasound volume data for each line of sight on the basis of the signal intensity of each voxel and the calculated opacity of the voxel, obtains pixel values, and forms an ultrasonic image on the basis of a plurality of pixel values for each line of sight.

Even in a case where ultrasound volume data include noise, the ultrasonic image processing device disclosed in the present specification can reduce an influence of the noise in a three-dimensional image obtained by performing volume rendering on the ultrasound volume data.

### BRIEF DESCRIPTION OF DRAWINGS

FIG. 1 is a schematic configuration diagram of an ultrasound diagnostic device according to the present embodiment;

FIG. 2 is a schematic configuration diagram of a three-dimensional image forming unit;

FIG. 3 illustrates a state in which ultrasound volume data is generated on the basis of a plurality of pieces of received frame data;

FIG. 4 is a conceptual diagram of ultrasound volume data;

FIG. 5 is a conceptual diagram illustrating a target voxel and neighboring voxels; and

FIG. 6 is an explanatory diagram of volume rendering.

### DESCRIPTION OF EMBODIMENTS

Hereinafter, the present embodiment will be described with reference to the drawings.

FIG. 1 is a schematic configuration diagram of an ultrasound diagnostic device 10 serving as an ultrasonic image processing device according to the present embodiment. The ultrasound diagnostic device 10 is installed in a medical institution such as a hospital and forms and displays an ultrasonic image on the basis of reception signals obtained by transmitting and receiving ultrasonic waves to and from a subject that is a living body. The subject includes, for example, a pregnant woman (particularly, a fetus).

In particular, the ultrasound diagnostic device 10 generates ultrasound volume data on the basis of reception signals obtained by transmitting and receiving ultrasonic waves to and from the subject and performs volume rendering on the ultrasound volume data, thereby forming a three-dimensional image that is an ultrasonic image. In particular, in a case where the subject is a fetus, ultrasound volume data are obtained by transmitting and receiving ultrasonic waves to and from the fetus, and the face and the like of the fetus are stereoscopically expressed in a three-dimensional image formed based on the ultrasound volume data. Such a three-dimensional image is provided for the pregnant woman or the like.

A probe 12, which is an ultrasonic probe, is a device that transmits ultrasonic waves and receives reflected waves. Specifically, the probe 12 is brought into contact with a body surface of the subject, transmits ultrasonic beams to the subject, and receives reflected waves reflected by tissue in the subject. The probe 12 includes a vibration element array including a plurality of vibration elements. Each vibration element included in the vibration element array receives supply of a transmission signal that is an electric signal from a transmission unit 14 described later, thereby generating an ultrasonic beam (transmission beam). Further, each vibration element included in the vibration element array receives a reflected wave from the subject, converts the reflected wave into a reception signal that is an electric signal, and transmits the reception signal to a receiving unit 16 described later.

In the present embodiment, the probe 12 is a one-dimensional ultrasonic probe in which the vibration element array is one-dimensionally arrayed. The probe 12 scans ultrasonic beams to form a beam scanning plane and scans ultrasonic beams on a plurality of beam scanning planes whose positions are slightly different from each other, thereby obtaining reception signals for generating ultrasound volume data. The probe 12 may be a two-dimensional ultrasonic probe in which the vibration element array is two-dimensionally arrayed. In the two-dimensional ultrasonic probe, two-dimensional ultrasonic beams can be formed by the two-dimensionally arrayed vibration element array, and thus reception signals for generating ultrasound volume data can be obtained.

The transmission unit 14 functions as a transmission beamformer. At the time of transmitting ultrasonic waves, the transmission unit 14 supplies a plurality of transmission signals to the probe 12 (specifically, the vibration element array) in parallel. Therefore, the probe 12 transmits ultrasonic beams.

The receiving unit 16 functions as a reception beamformer. At the time of receiving reflected waves, the receiving unit 16 receives a plurality of reception signals from the probe 12 (specifically, the vibration element array) in parallel. The receiving unit 16 performs processing such as integer addition processing on the plurality of reception signals, thereby generating received beam data. The received beam data have a plurality of signals (data) indicating signal intensity (indicated by value) of the reflected waves in a depth direction of the subject. A plurality of pieces of received beam data obtained from the beam scanning plane, which corresponds to one ultrasonic image, is used to form received frame data. In a case where the probe 12 is a two-dimensional ultrasonic probe, ultrasound volume data in which a plurality of pieces of received beam data is two-dimensionally arranged are formed.

A signal processing unit 18 performs various kinds of signal processing including detection processing, logarithmic amplification processing, gain correction processing, filter processing, or the like on each piece of the received beam data from the receiving unit 16.

A cine memory 20 stores a plurality of pieces of received frame data processed by the signal processing unit 18. The cine memory 20 is a first in first out (FIFO) buffer that outputs the received frame data from the signal processing unit 18 in the input order. In a case where the probe 12 is a two-dimensional ultrasonic probe, the ultrasound volume data is stored in the cine memory 20.

A three-dimensional image forming unit 22 generates a three-dimensional image by performing volume rendering processing on the ultrasound volume data based on the reception signals obtained by transmitting and receiving ultrasonic waves to and from the subject. Details of processing performed by the three-dimensional image forming unit 22 will be described later.

A display control unit 24 displays the three-dimensional image generated by the three-dimensional image forming unit 22 on a display 26 including, for example, a liquid crystal panel.

An input interface 28 includes, for example, a button, a trackball, or a touchscreen. The input interface 28 is for inputting a user's instruction to the ultrasound diagnostic device 10.

A memory 30 includes a hard disk drive (HDD), a solid state drive (SSD), an embedded multimedia card (eMMC), a read only memory (ROM), a random access memory (RAM), or the like. The memory 30 stores an ultrasonic image processing program for operating each unit of the ultrasound diagnostic device 10. The ultrasonic image processing program can also be stored in a computer-readable non-transitory storage medium such as a universal serial bus (USB) memory or a CD-ROM. The ultrasound diagnostic device 10 can read the ultrasonic image processing program from such a storage medium and execute the ultrasonic image processing program.

A control unit 32 includes at least one of a general-purpose processor (e.g. central processing unit (CPU)) and a dedicated processor (e.g. graphics processing unit (GPU), application specific integrated circuit (ASIC), field-programmable gate array (FPGA), or programmable logic device). The control unit 32 may not be configured by one processing device, but may be configured by cooperation of a plurality of processing devices existing at physically separated positions. The control unit 32 controls each unit of the ultrasound diagnostic device 10 according to the ultrasonic image processing program stored in the memory 30.

The transmission unit 14, the receiving unit 16, the signal processing unit 18, the three-dimensional image forming unit 22, and the display control unit 24 each include one or a plurality of processors, chips, electric circuits, and the like. Those units may be implemented by cooperation of hardware and software.

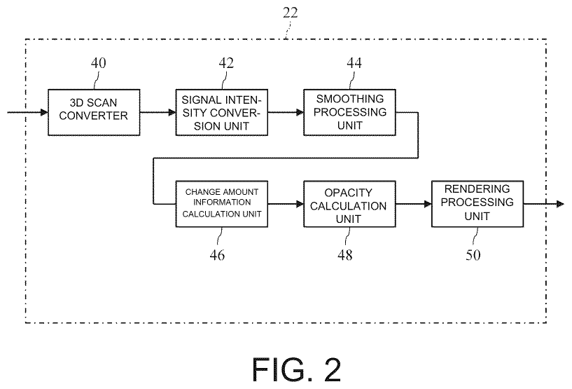

FIG. 2 is a schematic configuration diagram of the three-dimensional image forming unit 22. First, an overview of processing of the three-dimensional image forming unit 22 in the present embodiment will be described. As shown by the above Expression 1 in the conventional example, in volume rendering, an influence of signal intensity Xₙ of a voxel n on a calculated value Cₙ is larger as opacity α(Xₙ) of the voxel n is larger. Therefore, when the opacity of a voxel corresponding to noise (voxel having signal intensity caused by noise) is reduced, an influence of the noise on a calculated value (integrated value) of a ray is reduced; that is, the noise in a three-dimensional image is reduced.

The present embodiment focuses on the following feature: in ultrasound volume data, a voxel corresponding to noise has a smaller amount of change (difference) between the signal intensity of the voxel and the signal intensity of a neighboring voxel near the voxel (in other words, an amount of change between the signal intensity of a voxel corresponding to noise and the signal intensity of a neighboring voxel is less likely to increase). This is caused by a phenomenon that the difference in the signal intensity from the neighboring voxel increases (an edge is emphasized in terms of an image) because an amount of reflection of ultrasonic waves is large in a boundary portion of tissue in the subject (e.g. a surface of the face of a fetus), and the amount of reflection is small in a portion other than the boundary portion, whereas, for noise components, a plurality of voxels having similar signal intensity exist in a scattered state in many cases; i.e., in terms of an image, the image is blurred (has low contrast) in many cases. In the present embodiment, the opacity of each voxel is determined (corrected) on the basis of the amount of change in the signal intensity between the voxels. Therefore, the opacity of the voxel corresponding to the noise is reduced to reduce an influence of the noise in the three-dimensional image.

Hereinafter, details of volume rendering in the ultrasound diagnostic device 10 according to the present embodiment will be described together with details of processing performed by the three-dimensional image forming unit 22 with reference to FIGS. 2 to 4.

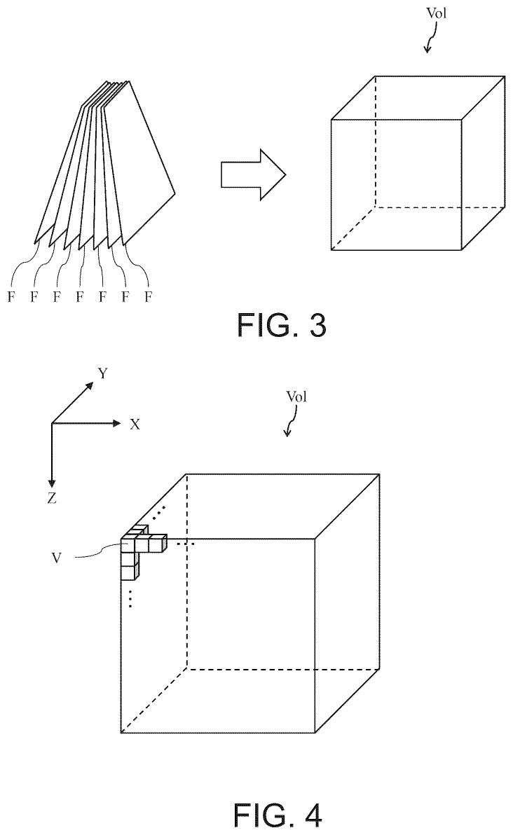

A 3D scan converter 40 generates ultrasound volume data on the basis of a plurality of pieces of frame data stored in the cine memory 20. Specifically, as illustrated in FIG. 3, the 3D scan converter 40 generates ultrasound volume data Vol by combining a plurality of pieces of received frame data F corresponding to a plurality of beam scanning planes whose positions are slightly different from each other. In a case where the probe 12 is a two-dimensional ultrasonic probe, the processing by the 3D scan converter 40 is unnecessary because the ultrasound volume data have already been stored in the cine memory 20.

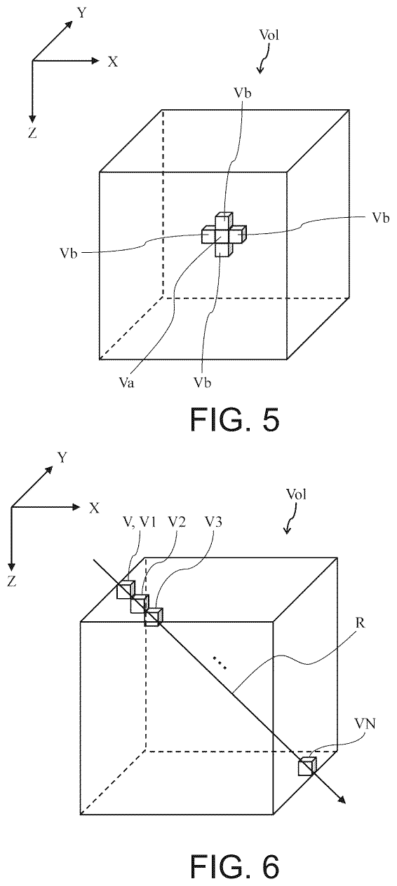

FIG. 4 is a conceptual diagram of the ultrasound volume data Vol. The ultrasound volume data Vol are data in which voxels V having data indicating signal intensity of reflected waves from the subject are three-dimensionally arranged. In FIG. 4 (and FIGS. 5 and 6), the X axis indicates, for example, an azimuth direction, the Y axis indicates, for example, a slice direction, and the Z axis indicates, for example, a depth direction. FIGS. 4 to 6 illustrate only some voxels V. The signal intensity of each voxel V is based on the signal intensity at each depth of the received beam data generated by the receiving unit 16 and processed by the signal processing unit 18.

A signal intensity conversion unit 42 performs processing of converting the signal intensity of each voxel V included in the ultrasound volume data Vol. Specifically, the signal intensity conversion unit 42 performs processing of setting the signal intensity of the voxel V having a signal intensity equal to or lower than a signal intensity threshold to 0. This makes it possible to simplify the subsequent processing in the three-dimensional image forming unit 22. The signal intensity threshold may be a predetermined value or may be settable by a user such as a doctor inputting an instruction through the input interface 28.

A smoothing processing unit 44 performs smoothing processing for smoothing the ultrasound volume data Vol. The smoothing processing is processing of applying, to each local region of the ultrasound volume data Vol, a smoothing filter including a small number of two-dimensional or three-dimensional pixels and having a weight set to each pixel, thereby making a gentler gradient of the signal intensity near an edge portion where a difference in the signal intensity between adjacent voxels V is large.

A change amount information calculation unit 46 calculates, for each voxel V forming the ultrasound volume data Vol, change amount information indicating an amount of change between the signal intensity of the voxel V (referred to as a target voxel Va) and the signal intensity of a neighboring voxel Vb near the target voxel Va.

FIG. 5 is a conceptual diagram illustrating a certain target voxel Va and its neighboring voxels Vb. In the present embodiment, voxels V adjacent to the target voxel Va in the X-axis, Y-axis, and Z-axis directions are neighboring voxels Vb (FIG. 5 does not illustrate neighboring voxels Vb adjacent to the target voxel Va in the Y-axis direction). Note that 26 voxels V around the target voxel Va not only in the X-axis, Y-axis, and Z-axis directions, but also in oblique directions may be set as the neighboring voxels Vb. However, in this case, an amount of calculation (described in detail later) for the amount of change of each voxel V increases. Thus, in the present embodiment, six voxels V in the X-axis, Y-axis, and Z-axis directions as viewed from the target voxel Va are set as the neighboring voxels Vb.

In the present embodiment, the voxels V adjacent to the target voxel Va are set as the neighboring voxels Vb, but is not necessarily limited thereto. The neighboring voxel Vb may be a voxel V not adjacent to the target voxel Va. For example, the neighboring voxel Vb may be a voxel V located a plurality of voxels away from the target voxel Va.

In the present embodiment, the change amount information, which indicates the amount of change in the signal intensity between the target voxel Va and the neighboring voxel Vb, is a difference directly showing the amount of change between the signal intensity of the target voxel Va and the signal intensity of the neighboring voxel Vb. However, the change amount information is not limited to the above difference and may be, for example, gradient intensity of the signal intensity between the target voxel Va and the neighboring voxel Vb, a gradient vector of the signal intensity between the target voxel Va and the neighboring voxel Vb, or a first-order (or second-order) differential value of the signal intensity of the target voxel Va in at least one of the X-axis, Y-axis, and Z-axis directions.

Specifically, in the present embodiment, the change amount information calculation unit 46 calculates the amount of change in the signal intensity between the target voxel Va and the neighboring voxel Vb as follows. First, the change amount information calculation unit 46 obtains a central difference in the signal intensity between the target voxel Va and the neighboring voxel Vb in each of the X-axis, Y-axis, and Z-axis directions by the following Expressions 3 to 5.

$$\\ \begin{matrix}[ Math . 3 ] & \\ dX ( x , y , z ) = \frac{( {X}_{n} ( x + L , y , z ) - {X}_{n} ( x - L , y , z ) )}{2 L} & ( Expression 3 )\end{matrix}$$

$$\\ \begin{matrix}dY ( x , y , z ) = \frac{( {X}_{n} ( x , y + L , z ) - {X}_{n} ( x , y - L , z ) )}{2 L} & ( Expression 4 )\end{matrix}$$

$$\\ \begin{matrix}dZ ( x , y , z ) = \frac{( {X}_{n} ( x , y , z + L ) - {X}_{n} ( x , y , z - L ) )}{2 L} & ( Expression 5 )\end{matrix}$$

In Expressions 3 to 5, X(x,y,z) represents the signal intensity of the voxel V at coordinates (x,y,z) in the ultrasound volume data Vol. L denotes an integer of 1 or more. That is, Expression 3 calculates the central difference in the signal intensity of the target voxel Va (the voxel V at coordinates (x,y,z)) in the X-axis direction (which is obtained by dividing a difference between the signal intensity of the neighboring voxel Vb at coordinates (x+L, y, z) and the signal intensity of the neighboring voxel Vb at coordinates (x-L, y, z) by 2L). Similarly, Expression 4 calculates the central difference in the signal intensity of the target voxel Va in the Y-axis direction, and Expression 5 calculates the central difference in the signal intensity of the target voxel Va in the Z-axis direction. When L=1, the neighboring voxel Vb is a voxel V adjacent to the target voxel Va, and, when L≥2, the neighboring voxel Vb is a voxel V not adjacent to the target voxel Va.

In the present embodiment, the change amount information calculation unit 46 sets the square root of the sum of squares of the central differences in the signal intensity calculated in the three directions as the amount of change in the signal intensity between the target voxel Va and the neighboring voxels Vb. That is, the change amount information calculation unit 46 calculates the amount of change in the signal intensity between the target voxel Va and the neighboring voxels Vb by the following Expression 6.

[Math. 4]

Vₙ(x,y,z)=\sqrt{dX(x,y,z)²+dY(x,y,z)²+dZ(x,y,z)²} (Expression 6)

As in Expression 6, the amount of change in the signal intensity between the voxel V and the neighboring voxels is denoted by Vₙ. By the above calculation, the amount of change Vₙ in the signal intensity between the voxel and the neighboring voxels is calculated for all the voxels V forming the ultrasound volume data Vol.

The amount of change Vₙ in the signal intensity between the voxel V and the neighboring voxels may be calculated by another method as described below.

For example, the change amount information calculation unit 46 may obtain Vₙ by using one of the central differences in the signal intensity between the target voxel Va and the neighboring voxels Vb in the X-axis, Y-axis, and Z-axis directions. For example, the change amount information calculation unit 46 may set a maximum value of the central differences in the signal intensity in the respective axial directions as Vₙ as shown by the following Expression 7 or may set a minimum value of the central differences in the signal intensity in the respective axial directions as Vₙ as shown by the following Expression 8.

$$\\ \begin{matrix}[ Math . 5 ] & \\ {V}_{n} ( x , y , z ) = {max}_{} ( dX ( x , y , z ) , dY ( x , y , z ) , dZ ( x , y , z ) ) & ( Expression 7 )\end{matrix}$$

$$\\ \begin{matrix}{V}_{n} ( x , y , z ) = {min}_{} ( dX ( x , y , z ) , dY ( x , y , z ) , dZ ( x , y , z ) ) & ( Expression 8 )\end{matrix}$$

The change amount information calculation unit 46 may obtain Vₙ by using two of the central differences in the signal intensity between the target voxel Va and the neighboring voxels Vb in the X-axis, Y-axis, and Z-axis directions. For example, as shown by the following Expression 9, the change amount information calculation unit 46 may set the square root of the sum of squares of two large values among the central differences in the signal intensity in the three axial directions as Vₙ.

[Math. 6]

When dX(x,y,z)>dZ(x,y,z) and, dY(x,y,z)>dZ(x,y,z)

[symbol]

Vₙ(x,y,z)=\sqrt{dX(x,y,z)²+dY(x,y,z)²} [symbol] (Expression 9)

### AND

The change amount information calculation unit 46 may set an average value of the central differences in the signal intensity between the target voxel Va and the neighboring voxels Vb in the X-axis, Y-axis, and Z-axis directions as Vₙ, as shown by the following Expression 10.

$$\\ \begin{matrix}[ Math . 7 ] & \\ {V}_{n} ( x , y , z ) = {ave}_{} ( dX ( x , y , z ) , dY ( x , y , z ) , dZ ( x , y , z ) ) & ( Expression 10 )\end{matrix}$$

When calculating the central differences in the signal intensity between the target voxel Va and the neighboring voxels Vb in the X-axis, Y-axis, and Z-axis directions, the change amount information calculation unit 46 may perform the calculation while performing smoothing by using a Sobel filter. Alternatively, the difference in the signal intensity between the target voxel Va and the neighboring voxel Vb in each axial direction may be a difference in the signal intensity between the target voxel Va and one neighboring voxel Vb in each axial direction, instead of the central difference.

Returning to FIG. 2, an opacity calculation unit 48 calculates the opacity of each voxel V on the basis of the change amount information (the amount of change Vₙ in the present embodiment) regarding each voxel V calculated by the change amount information calculation unit 46. Specifically, the opacity calculation unit 48 performs calculation such that the opacity of the voxel V is reduced with reduction in the amount of change in the signal intensity between the voxel V and the neighboring voxel, the amount of change being indicated by the change amount information regarding the voxel V.

Based on the change amount information regarding each voxel V, the opacity calculation unit 48 calculates, for each voxel V, a correction parameter for correcting the opacity α(Xₙ) predetermined for each voxel V included in the ultrasound volume data Vol. As described later, the opacity α(Xₙ) of each voxel is corrected on the basis of the opacity α(Xₙ) of each voxel and the correction parameter. Thus, calculation of the correction parameter by the opacity calculation unit 48 is synonymous with calculation of the opacity of each voxel by the opacity calculation unit 48.

In the present embodiment, the change amount information is represented by the amount of change Vₙ in the signal intensity between each voxel V and its neighboring voxel. Thus, the opacity calculation unit 48 calculates the correction parameter on the basis of the amount of change Vₙ. Specifically, a correction parameter g(Vₙ) of each voxel V is calculated by the following Expression 11.

$$\\ \begin{matrix}[ Math . 8 ] & \\ g ( {V}_{n} ) = \{ \begin{matrix}1. & ( {V}_{max} \leq {V}_{n} ) \\ \frac{{V}_{n}}{{V}_{max}} & ( {V}_{min} \leq {V}_{n} < {V}_{max} ) \\ 0. & ( {V}_{n} < {V}_{min} )\end{matrix} & ( Expression 11 )\end{matrix}$$

As shown by Expression 11, the opacity calculation unit 48 sets the correction parameter g(Vₙ) to 1 in a case where the amount of change Vₙ of the voxel V is equal to or larger than a predetermined maximum value Vₘₐₓ, sets the correction parameter g(Vₙ) to Vₙ/Vₘₐₓ in a case where the amount of change Vₙ of the voxel V is less than the maximum value Vₘₐₓ and is equal to or larger than a predetermined minimum value Vₘᵢₙ, and sets the correction parameter g(Vₙ) to 0 in a case where the amount of change Vₙ of the voxel V is less than the predetermined minimum value Vₘᵢₙ. That is, the opacity calculation unit 48 calculates the correction parameter g(Vₙ) by normalizing the amount of change Vₙ to a value between the minimum value Vₘᵢₙ and the maximum value Vₘₐₓ.

Although the correction parameter g(Vₙ) shown by the above Expression 11 represents linear correction, the correction parameter g(Vₙ) may represent non-linear correction. In that case, the opacity calculation unit 48 calculates the correction parameter g(Vₙ) for each voxel V by, for example, the following Expression 12.

$$\\ \begin{matrix}[ Math . 9 ] & \\ g ( {V}_{n} ) = \{ \begin{matrix}1. & ( {V}_{max} \leq {V}_{n} ) \\ f ( {V}_{n} ) & ( {V}_{min} \leq {V}_{n} < {V}_{max} ) \\ 0. & ( {V}_{n} < {V}_{min} )\end{matrix} & ( Expression 12 )\end{matrix}$$

In Expression 12, a transform function f(Vₙ) is an expression normalized from Vₘᵢₙ to Vₘₐₓ and is expressed by, for example, the following Expression 13.

$$\\ \begin{matrix}[ Math . 10 ] & \\ ↵ & ( Expression 13 )\end{matrix}$$

$$\\ f ( {V}_{n} ) = \frac{s ( {V}_{n} ) - s ( {V}_{min} )}{s ( {V}_{max} ) - s ( {V}_{min} )} .$$

In Expression 13, s(Vₙ) may be a sigmoid function and is expressed by the following Expression 14.

$$\\ \begin{matrix}[ Math . 11 ] & \\ ↵ & ( Expression 14 )\end{matrix}$$

$$\\ s ( {V}_{n} ) = \frac{1}{1 + exp ( - gain * n ( {V}_{n} ) )}$$

In Expression 14, gain is a parameter for determining steepness of a sigmoid curve. The parameter gain may be adjustable by the user or may be a predetermined fixed value. Further, n(Vₙ) is a normalization expression so as to obtain 1 when Vₙ is Vₘₐₓ and obtain −1 when Vₙ is Vₘᵢₙ and is expressed by the following Expression 15.

$$\\ \begin{matrix}[ Math . 12 ] & \\ n ( {V}_{n} ) = 2. * ( \frac{{V}_{n} - {V}_{min}}{{V}_{max} - {V}_{min}} - 0.5 ) & ( Expression 15 )\end{matrix}$$

Then, for each voxel V, the opacity calculation unit 48 sets the product of the opacity α(Xₙ) predetermined for the voxel V and the correction parameter g(Vₙ) calculated for the voxel V as corrected opacity of the voxel V. That is, the corrected opacity of each voxel V is shown by the following Expression 16.

[Math. 13]

α(Xₙ)*g(Vₙ) (Expression 16)

In a case where the correction parameter g(Vₙ) is 1, the opacity α(Xₙ) is not corrected. In a case where the correction parameter g(Vₙ) is 0, the opacity of the voxel V is 0. That is, the signal intensity of the voxel V is ignored at all in volume rendering in a rendering processing unit 50 described later. In a case where the correction parameter g(Vₙ) is Vₙ/Vₘₐₓ, Vₙ/Vₘₐₓ is a value smaller than 1 because Vₘᵢₙ≤Vₙ≤Vₘₐₓ. Therefore, the opacity of the voxel is smaller than the predetermined value. This reduces an influence of the signal intensity of the voxel V in the volume rendering, as compared with a case where the opacity before being corrected is used.

In the present embodiment, the maximum value that the correction parameter g(Vₙ) can take is 1, but the correction parameter g(Vₙ) may take a value of 1 or more. In a case where the correction parameter g(Vₙ) is 1 or more, the opacity of the voxel V is corrected to be large, and the influence of the signal intensity of the voxel V is emphasized in the volume rendering.

At least one of the maximum value Vₘₐₓ and the minimum value Vₘᵢₙ included in Expression 11 or Expression 12 may be changeable in response to a user instruction. When the maximum value Vₘₐₓ decreases, the number of voxels V whose opacity is not corrected increases, and, when the maximum value Vₘax increases, the number of voxels V whose opacity is corrected increases. Meanwhile, when the minimum value Vₘᵢₙ decreases, the number of voxels V having the opacity of 0 (completely ignored in the volume rendering) decreases, and, when the minimum value Vₘᵢₙ increases, the number of voxels V having the opacity of 0 increases. The user can adjust at least one of the maximum value Vₘₐₓ and the minimum value Vₘᵢₙ so as to obtain a desired three-dimensional image in the volume rendering.

The rendering processing unit 50 sets a line of sight (ray R) passing through the ultrasound volume data Vol from a certain viewpoint (see FIG. 6). Then, integration processing is performed for each ray on the basis of the signal intensity Xₙ of each voxel V (in FIG. 6, voxels V1, V2, . . . VN) on the ray R and the corrected opacity α(Xₙ)*g(Vₙ). Thus, a calculated value is obtained. Specifically, the rendering processing unit 50 performs, for each ray, integration processing shown by the following Expression 17 obtained by replacing the opacity α(Xₙ) of each voxel in the above Expression 1 according to the conventional example with the corrected opacity α(Xₙ)*g(Vₙ).

[Math. 14]

Cₙ=Cₙ₋₁+(1−αₙ₋₁)*{α(Xₙ)*g(Vₙ)}*Xₙ (Expression 17)

The integrated value αₙ of the opacity up to the voxel n on the ray R is shown by the following Expression 18 obtained by replacing the opacity α(Xₙ) of each voxel in the above Expression 2 according to the conventional example with the corrected opacity α(Xₙ)*g(Vₙ).

[Math. 15]

αₙ=αₙ₋₁(1−αₙ₋₁)*{α(Xₙ)*g(Vₙ)} (Expression 18)

As shown by Expressions 17 and 18, the rendering processing unit 50 performs volume rendering on the ultrasound volume data Vol for each line of sight on the basis of the signal intensity Xₙ of each voxel V and the opacity α(Xₙ)*g(Vₙ) of each voxel V calculated (corrected) by the opacity calculation unit 48, obtains pixel values, and forms a three-dimensional image that is an ultrasonic image on the basis of a plurality of pixel values for each line of sight.

As described above, the voxel V corresponding to noise (the voxel V having the signal intensity caused by noise) has a feature that the amount of change Vₙ in the signal intensity from the neighboring voxel is reduced. According to the present embodiment, the opacity calculation unit 48 calculates the correction parameter g(Vₙ) having a smaller value as the amount of change Vₙ is smaller, as shown by the above Expression 11 or Expressions 12 to 15. That is, the corrected opacity α(Xₙ)*g(Vₙ) of the voxel V is reduced as the amount of change Vₙ is reduced. Therefore, an influence of the signal intensity (noise components) of the voxel V corresponding to the noise on the three-dimensional image is reduced in the volume rendering.

Meanwhile, in a boundary portion of tissue to be shown in the three-dimensional image (e.g. a surface of the face of a fetus), the amount of change Vₙ in the signal intensity from the neighboring voxel increases. Thus, the correction parameter g(Vₙ) has a value of 1 or a value close to 1. Therefore, the opacity of the voxel V corresponding to the boundary portion of the tissue is not corrected (reduced) much. Thus, the boundary portion of the tissue is less likely to be blurred or luminance is less likely to be reduced in the three-dimensional image.

Although the ultrasonic image processing device according to the present disclosure has been described above, the ultrasonic image processing device according to the present disclosure is not limited to the above embodiment, and various modifications can be made without departing from the gist thereof.

For example, in the present embodiment, the ultrasonic image processing device is the ultrasound diagnostic device 10. However, the ultrasonic image processing device is not limited to the ultrasound diagnostic device 10 and may be another computer. In this case, a computer serving as the ultrasonic image processing device exerts the function of the three-dimensional image forming unit 22. Specifically, the computer serving as the ultrasonic image processing device receives a plurality of pieces of received frame data or ultrasound volume data from an ultrasound diagnostic device and performs calculation of change amount information regarding each voxel V, calculation of the opacity of each voxel, and volume rendering on the plurality of pieces of received frame data or the ultrasound volume data.

1. An ultrasonic image processing device comprising:
a change amount information calculation unit that calculates change amount information indicating an amount of change between signal intensity of a target voxel and signal intensity of a neighboring voxel near the target voxel for each voxel forming ultrasound volume data obtained by transmitting and receiving ultrasonic waves to and from a subject;
an opacity calculation unit that calculates opacity of each voxel on the basis of the change amount information regarding the voxel such that the opacity of the voxel is reduced with reduction in the amount of change in the signal intensity between the voxel and the neighboring voxel, the amount of change being indicated by the change amount information regarding the voxel; and
a rendering processing unit that performs volume rendering on the ultrasound volume data for each line of sight on the basis of the signal intensity of the voxel and the calculated opacity of the voxel, obtains pixel values, and forms an ultrasonic image on the basis of a plurality of pixel values for each line of sight.

2. The ultrasonic image processing device according to claim 1, wherein
the opacity calculation unit calculates a correction parameter based on the amount of change for each voxel and corrects the opacity of the voxel on the basis of opacity predetermined for the voxel and the correction parameter.

3. The ultrasonic image processing device according to claim 2, wherein:
the opacity calculation unit calculates the correction parameter by normalizing the amount of change to a value between a predetermined minimum value and a predetermined maximum value; and
at least one of the maximum value and the minimum value is changeable in response to a user instruction.

4. The ultrasonic image processing device according to claim 1, wherein
the neighboring voxel is a voxel not adjacent to the target voxel in the ultrasound volume data.

5. The ultrasonic image processing device according to claim 1, wherein
the ultrasound volume data are obtained by transmitting and receiving an ultrasonic wave to and from a fetus.

6. A computer-readable non-transitory storage medium storing a command executable by a computer, the command causing the computer to execute:
a change amount information calculation step of calculating change amount information indicating an amount of change between signal intensity of a target voxel and signal intensity of a neighboring voxel near the target voxel for each voxel forming ultrasound volume data obtained by transmitting and receiving ultrasonic waves to and from a subject;
an opacity calculation step of calculating opacity of each voxel on the basis of the change amount information regarding the voxel such that the opacity of the voxel is reduced with reduction in the amount of change in the signal intensity between the voxel and the neighboring voxel, the amount of change being indicated by the change amount information regarding the voxel; and
a rendering processing step of performing volume rendering processing on the ultrasound volume data for each line of sight on the basis of the signal intensity of each voxel and the calculated opacity of the voxel, obtaining pixel values, and forming an ultrasonic image on the basis of a plurality of pixel values for each line of sight.
